(19) United States
(12) Patent Application Publication
(10) Pub. No.: US 2024/0358495 A1
(43) Pub. Date: Oct. 31, 2024

(54) ENDOLEAK DEVICE

(71) Applicants: Michael SIEGENTHALER, New Albany, OH (US); Andrew Howard SCHULICK, Bethesda, MD (US)

(72) Inventors: Michael SIEGENTHALER, New Albany, OH (US); Andrew Howard SCHULICK, Bethesda, MD (US)

(21) Appl. No.: 18/277,392

(22) PCT Filed: Feb. 14, 2022
(86) PCT No.: PCT/US2022/016290

§ 371 Date: Aug. 16, 2023

Related U.S. Application Data
(60) Provisional application No. 63/149,885, filed on Feb. 16, 2021.

(51) Int. Cl. A61F 2/06; A61F 2/01; A61F 2/07; A61M 1/36

(52) U.S. Cl. CPC A61F 2/064; A61F 2/012; A61F 2/07; A61M 1/3655; A61F 2002/068; A61F 2002/072

(57) ABSTRACT

A device is provided to be implanted to create a temporary or permanent connection between two hollow structures. The hollow structures may be, for example, an aortic or large vessel aneurysm sac and a large adjacent vein.

### RELATED APPLICATIONS

This application claims the benefit of priority to U.S. provisional application No. 63/149,885, filed Feb. 16, 2021 and titled “Endoleak Device,” the entire contents of which are incorporated herein by reference.

### BACKGROUND

### Field

The present disclosure is directed to a device and method to create and maintain a connection between adjacent hollow structures. The utility of the device is exemplified when creating a communication between 2 vascular channels. Specifically, such as a large artery and adjacent vein or one cardiac chamber to another. Specifically, the pressurized and expanding residual sac of a previously stented abdominal aortic aneurysm (AAA), and the adjacent Inferior Vena Cava (IVC).

In this exemplary circumstance, creating a communication between the adjacent IVC, and the AAA sac exhibiting endoleak, allows the higher pressure AAA side to vent across a gradient to the lower pressure IVC; the relief in pressure from the residual aneurysm sac decreases the possibility of continued AAA sac growth and aneurysm rupture.

In addition to its function in pressure relief, the device is designed to act as a conduit to aid in transluminal or percutaneous interventions from either direction and can aid in defining difficult to diagnose sources of endoleaks.

### Description of the Related Art

An arterial aneurysm is an abnormal dilation of an artery resulting in the weakening of the vessel wall. The progressive thinning of the wall, akin to inflating a balloon, can lead to catastrophic rupture, resulting exsanguination and death. Of the many types of arterial aneurysms, AAAs are the most common. Arterial blood pressure is one of the main driving forces resulting in arterial expansion. Because of the risk of rupture, when AAAs are detected, they are carefully monitored, and when certain criteria are met, the most important being size, the aneurysms are treated. The most common aneurysm treatment in modern practice is performed using endovascular stent grafting.

In endovascular aortic stent grafting, the aneurysm is excluded from blood flow by use of a fluid impermeable, fabric covered, cylindrical stent to span the length of the diseased artery. In general the upstream and downstream seal zones of the stent graft are comprised of segments of normal caliber artery. Because the stent graft is fluid impermeable, the aneurysm is excluded from arterial blood flow—the concept being, no flow no pressure, no pressure no growth, no growth no rupture. In practice, however, any of the seal zones, as well as the aforementioned side branches, can result in endoleak, or continued blood flow into the aneurysm sac, which results in sac pressurization.

Endoleaks are subdivided into several types. Type 1 endoleaks are caused by failure of the seal zones between the stent and the native normal artery to prevent blood flow from entering into the aneurysm (FIG. 1a, 1b; 1a, 1b). Type 2 endoleaks, the most common form, are caused by back bleeding from side branches of the excluded aorta (FIG. 1b; 6,7). Type 3 endoleaks are caused by failure of the overlapped connection between multiple stent grafts to seal (FIG. 1a; 4). Type 4 endoleaks are cause by fabric porosity and are generally self-limited. To check for endoleak, stent graft repaired AAAs are followed with interval contrast CT scanning.

If endoleaks are detected after repair, continued follow up, with serial contrast CT scan imaging is indicated. Type 1 and 3 endoleaks usually require early re-operative repair, because they represent the transmission of direct systemic arterial pressure to the aneurysm. Type 2 endoleaks, which are by far the most common, can often be treated expectantly. This is because branch artery back bleeding is thought to transmit less pressure to the residual sac. Intervention for type 2 endoleaks is indicated, however, if serial scanning demonstrates that the residual sac of the AAA exhibits growth. Interventions to treat endoleaks, especially the most common Type 2, can be technically challenging. Treatment is generally attempted via a less invasive endovascular approach. This is particularly difficult and time-consuming for Type 2 endoleaks, as the operator attempts to reach the aneurysm sac through a circuitous, maze-like, network of collateral arteries feeding into the aneurysm sac. If the aneurysm cannot be reached, the next step is higher risk percutaneous direct puncture of the residual aneurysm sac to access the feeding branches. In either approach, once the sac is accessed, the operator then attempts to seal the leak by obliterating the side branches using endovascular methods such as coils or liquid embolization. Often multiple interventions are required because sealing some branches can result in pressurization of other branches causing new endoleaks where none seemed to be present previously. Occasionally satisfactory resolution of endoleak cannot be achieved despite multiple interventions. In addition, the AAA sac may continue to grow despite apparent resolution of endoleak, or even in the absence of a detectable endoleak. These Type 2 endoleak re-interventions to seal off side branches represent an “Achilles Heel” in the treatment of AAAs, and are a source of frustration for both operator and patient. More practically, the patient incurs continued risk from ionizing radiation for diagnosis and treatment, as well as risk of aneurysm rupture from an incompletely treated AAA. In the end, some patients whose aneurysms grow dangerously large require old fashioned open operative repair to treat the endograft failure.

The current device and method provides a paradigmatically different form of treatment, and is especially effective against the most common Type 2 endoleak. The device creates and maintains a direct, potentially permanent, connection between the aortic aneurysm sac and the adjacent IVC, allowing arterial pressure to be vented from the aneurysm sac to the lower pressure venous side, akin to arteriovenous fistulas used in dialysis access. By eliminating excessive pressure buildup in the aneurysm sac, risk of rupture is mitigated. In addition to its function in pressure relief, the design of the device also allows it to serve as an easy to establish access conduit between the two vessels for much easier treatment of any source of endoleak and can be re-accessed in the future, should future interventions be required.

### SUMMARY

Present disclosure is directed to the endovascular treatment of AAA and other aortic and large vessel aneurysms. The majority of these aneurysms are treated, when indicated, using an endovascular approach with fluid impermeable fabric covered stents (stent grafts). In the AAA segment, several configurations are possible for treatment, but the most commonly employed consists of a bifurcated stent graft with a modular docking system resulting in a “pants-like” configuration (FIG. 1a). In this approach, the aneurysm itself is not removed. Instead the stent graft forms an internal bypass, spanning the diseased segment of artery. Notably, side branches emanating from the treated segment are generally left unmolested (FIG. 1b). Whereas prior to stenting, these native aortic side branches represent egress channels, when the AAA is treated with a covered stent, which eliminates arterial pressure in the aneurysm, blood in these side branches can flow in reverse resulting in unwanted blood return to the aneurysm sac (endoleak).

Even after “successful” endovascular AAA repair, up to 30% of AAA sacs demonstrate endoleak. The most common form of endoleak is back bleeding into the residual sac from branch vessels of the abdominal aorta. Frequently, this arterial back bleeding results in enough pressure being transmitted to the residual AAA sac that the aneurysm continues to grow despite stent graft treatment. In these circumstances endoleak treatment is necessary, often requiring multiple, complex, time consuming interventions targeted at the side branches. Occasionally conversion of endovascular repair to formal open operative repair is ultimately necessary to mitigate risk of rupture inherent with continued aneurysm growth.

The device of the present disclosure greatly simplifies treatment of endoleak, offering direct intervention of the AAA sac itself; this represents a paradigmatically novel approach to endoleak intervention. Specifically, the present disclosure provides:

In an exemplary aspect, a device is configured to be implanted to create a temporary or permanent connection between two hollow structures, for example, between an aortic or large vessel aneurysm sac and a large adjacent vein.

In an exemplary aspect, the implantable device includes a stent that is either balloon expandable or is self-expandable

In an exemplary aspect, the implantable device is either an open cell stent without covering, or the stent is partially or fully covered with fabric or film.

In an exemplary aspect, there is a one or a plurality of filters at one or both ends of the device or within the cylindrical portion of the device

In an exemplary aspect, components of the device can be joined to bridge different distances between the aneurysm sac and the vein.

In an exemplary aspect, one or both ends of the device are conical, funnel shaped, tapered, or flanged.

In an exemplary aspect, a conical or umbrella shaped end of the device is attachable to either end or both ends, i.e., the aortic sac or the venous side

In an exemplary aspect, a flow-reducing valve or a plurality of valves is present at one or both ends of the stent of the device, or within the stent.

In an exemplary aspect, the stent of the device, or any of its component parts have a textured surface, have one or a plurality of barb rows, have one or a plurality of donuts, toroids, or undulations, or other surface features that offer resistance to movement or slippage.

In an exemplary aspect, the cylindrical member of the device is comprised in part or in whole of a stent or a stent graft.

In an exemplary aspect, the filter of the device may be flat, dome shaped or conical, oriented antegrade or retrograde to flow, and may be integrated or detachable.

In an exemplary aspect, the filter of the device is open cell, wire mesh or partially or completely fabric covered.

In an exemplary aspect, the implantable incorporates at least one conical, tapered, or flared end to facilitate future access to the aneurysm sac.

In an exemplary aspect, the implantable device is configured to be closed or partially closed with a plug should the arterio-venous connection require reduced flow or elimination.

In an exemplary aspect, the device includes a plug for the purpose of closing or reducing flow through the device. The plug may be shaped to allow future re-access to the spanned structures. The shape may include a funnel or conical shape on one or both sides, tapering to a small, wire accessible central lumen.

In an exemplary aspect, a method of creating a temporary or permanent connection between two hollow stuctures includes creating an arterio-venous fistula connection between a large vein such as the inferior vena cava, and the abdominal aorta or an aortic aneurysm sac. The method is applicable from either a transvenous approach, a direct percutaneous approach through the aneurysm sac, or an approach involving access through branch arteries or veins.

In an exemplary aspect, the device or its associated plug may be comprised of a variety or plurality of materials, including but not limited to metals, fabrics, plastics, films. The materials may be designed to be permanent, or temporary, some may be fully or partially dissolvable. Parts of the device or plug may utilize temporary or permanent attachment features such as hooks, screws, wires, magnets, glues, and resins.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1a illustrates the anatomy of an abdominal aortic aneurysm after a modular stent graft implantation and its anatomic juxtaposition of the inferior vena cava to the aortic aneurysm sac, according to exemplary embodiments of the disclosure;

FIG. 1b illustrates the same anatomy and illustrates the potential sources of endoleaks, according to exemplary embodiments of the present disclosure;

FIG. 2 illustrates one potential version of the device, which is deployed and connects the IVC and the aortic aneurysm sac, according to exemplary aspects of the present disclosure;

FIG. 3a illustrates the device, which has an open cell configuration at its ends and has a covered middle section, according to exemplary aspects of the present disclosure;

FIG. 3b illustrates the device with a fabric covered configuration, according to exemplary aspects of the present disclosure;

FIG. 3c illustrates an uncovered device with bare metal stent scaffolding exposed, according to exemplary aspects of the present disclosure;

FIG. 4a is a view of a partially deployed balloon expandable device connecting a large vein such as the inferior vena cava or another adjacent large vein to the aneurysm sac, according to exemplary aspects of the present disclosure;

FIG. 4b illustrates the fully expanded device, according to exemplary aspects of the present disclosure;

FIG. 5a is a first illustration of a device with a filter and reverse barbs that include an integrated screw off wire-release mechanism, according to exemplary aspects of the present disclosure;

FIG. 6b is a second illustration of a device with a filter and reverse barbs that include an integrated screw off wire-release mechanism, according to exemplary aspects of the present disclosure;

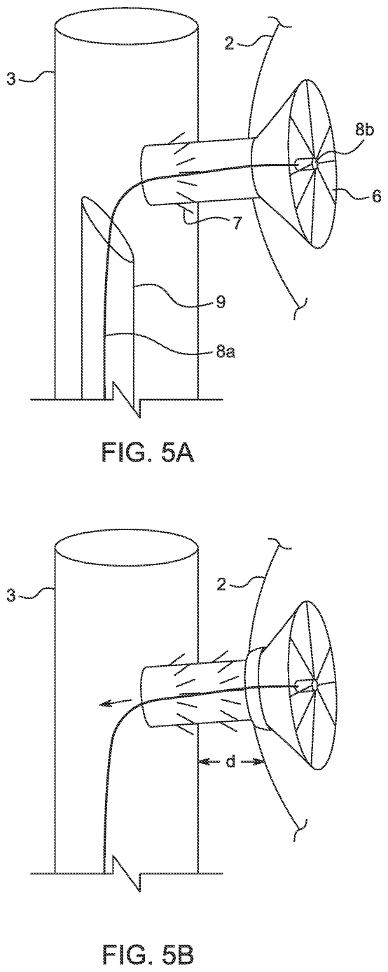

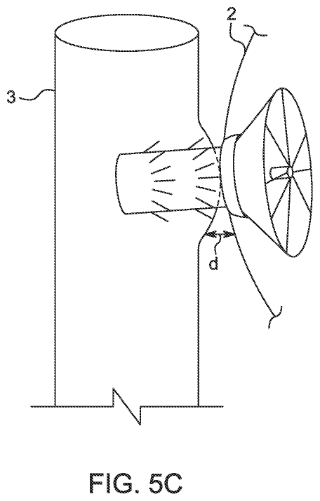

FIG. 5c is a third illustration of a device with a filter and reverse barbs that include an integrated screw off wire-release mechanism, according to exemplary aspects of the present disclosure;

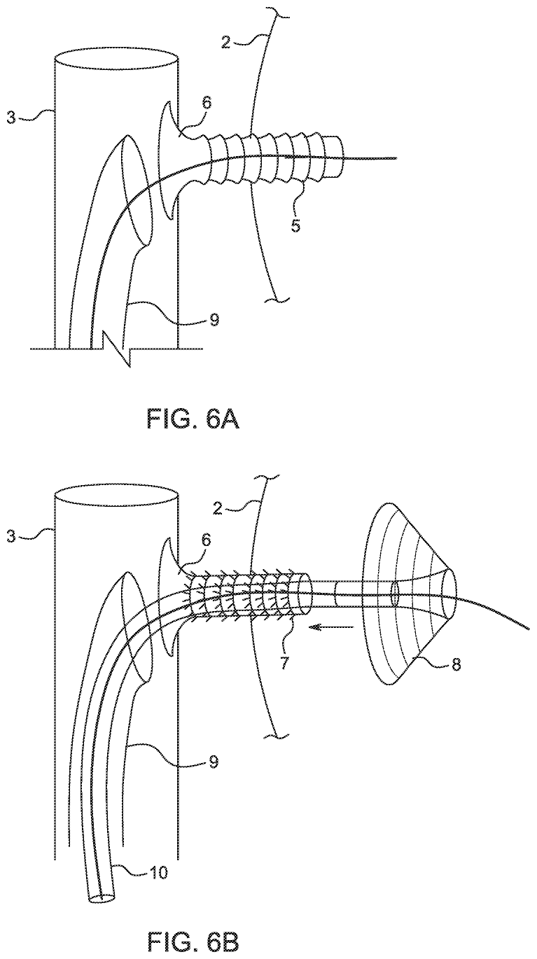

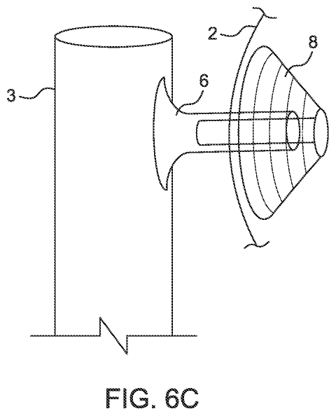

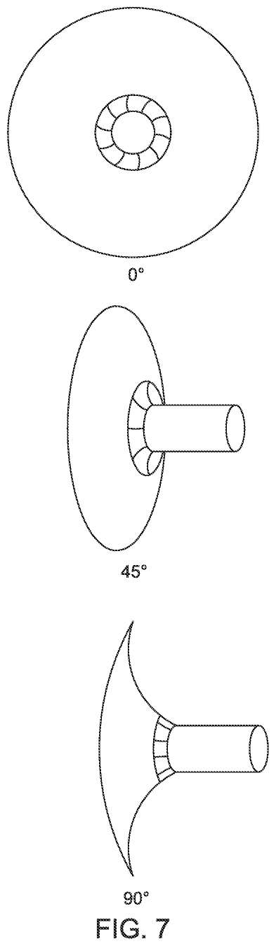

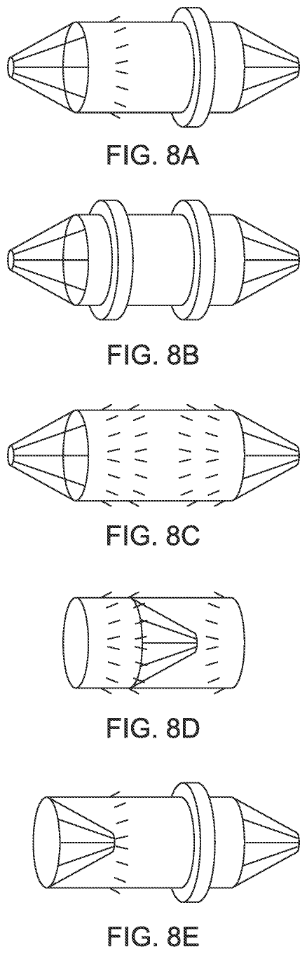

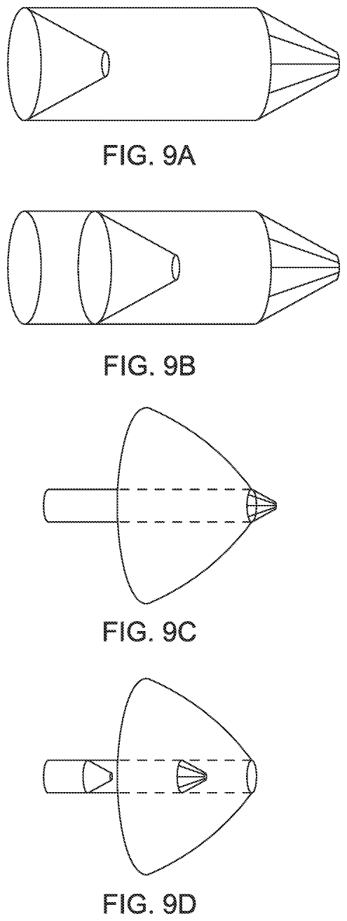

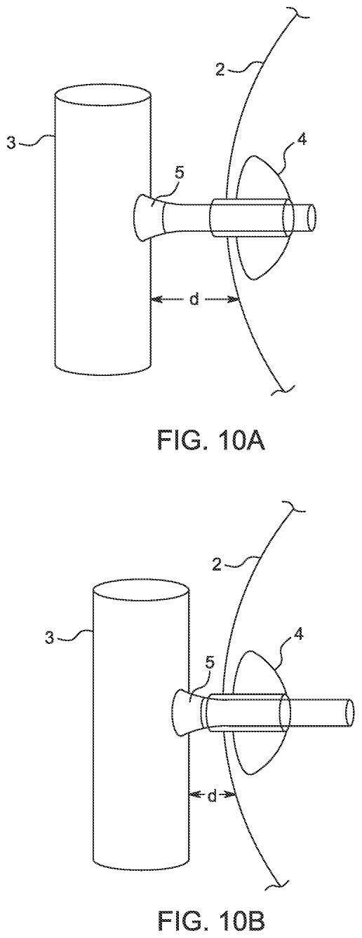

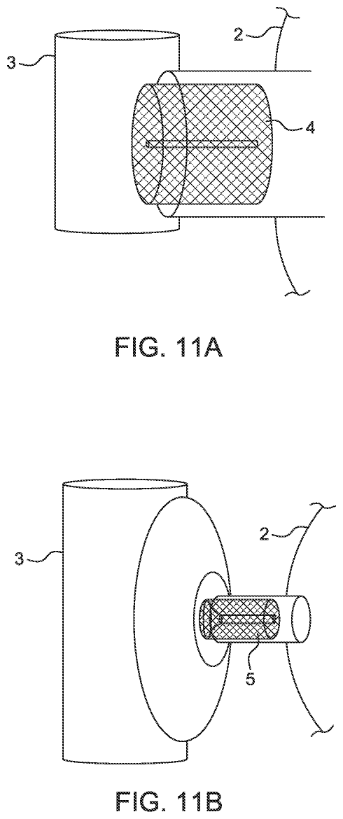

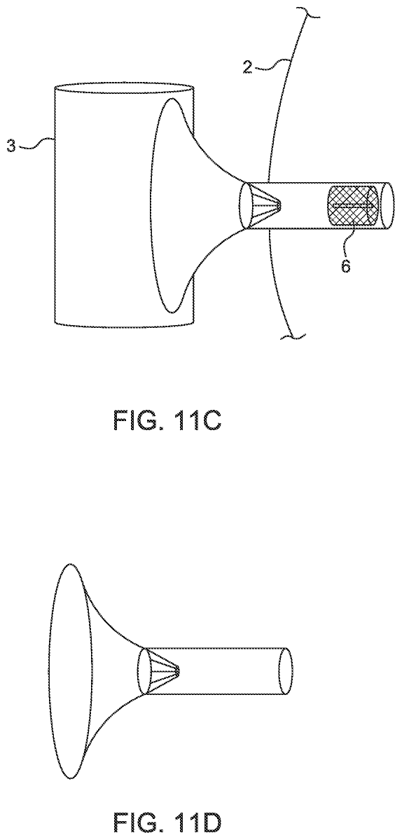

FIG. 6a is a first illustration of a device configuration with two components, which initially a device with a flat flange on the venous side and reverse is deployed from the IVC side, according to exemplary aspects of the present disclosure;

FIG. 6b is a second illustration of a device configuration with two components, which initially a device with a flat flange on the venous side and reverse is deployed from the IVC side, according to exemplary aspects of the present disclosure;

FIG. 6c is a third illustration of a device configuration with two components, which initially a device with a flat flange on the venous side and reverse is deployed from the IVC side, according to exemplary aspects of the present disclosure;

FIG. 7 illustrates ‘on face’ (0°, a 45° and a side-view (90°) of a funnel shape end of the device which can be placed on the venous or aneurysm sac side, according to exemplary aspects of the present disclosure;

FIG. 8a illustrates a device iteration with a filter on both ends of a device with an integrated sealing ring, according to exemplary aspects of the present disclosure;

FIG. 8b illustrates a device with a filter and sealing ring on both ends, according to exemplary aspects of the present disclosure:

FIG. 8c illustrates rows of reversed barbs and filters are present in a device according to exemplary aspects of the present disclosure;

FIG. 8d illustrates the device with a conical filter inside the cylindrical portion of the device, according to exemplary aspects of the present disclosure;

FIG. 8e illustrates another itineration of the device with a conical filter inside the device, but in a different position, according to exemplary aspects of the present disclosure;

FIG. 9a illustrates a configuration of the one and two component device with a filter and flow reducing valves, according to exemplary aspects of the present disclosure;

FIG. 9b illustrates another configuration of the one and two component device with a filter and flow reducing valves, according to exemplary aspects of the present disclosure;

FIG. 9c illustrates a further configuration of the one and two component device with a filter and flow reducing valves, according to exemplary aspects of the present disclosure;

FIG. 9d illustrates a still further configuration of the one and two component device with a filter and flow reducing valves, according to exemplary aspects of the present disclosure;

FIG. 10a illustrates a 2-component device, where the aortic sac component with an umbrella shaped end gets deployed first and the second component with a long flange is then placed inside component, according to exemplary aspects of the present disclosure;

FIG. 10b illustrates another 2-component device, where the aortic sac component with an umbrella shaped end gets deployed first and the second component with a long flange is then placed inside component, according to exemplary aspects of the present disclosure;

FIG. 11a illustrates a cylindrical plug with a central lumen for closure of the cylindrical portion of the device, according to exemplary aspects of the present disclosure;

FIG. 11b illustrates a cylindrical plug with a funnel shape towards a central lumen for closure of the cylindrical channel of the device, according to exemplary aspects of the present disclosure;

FIG. 11c illustrates a cylindrical plug with a central lumen for closure of the cylindrical portion of the device, according to exemplary aspects of the present disclosure; and

FIG. 11d illustrates a device with reversal of the filter direction, according to exemplary aspects of the present disclosure.

### DETAILED DESCRIPTION

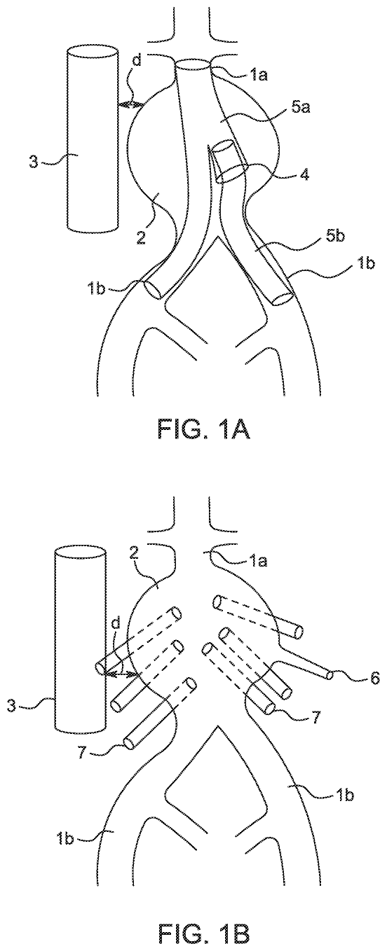

Referring now to the drawings, wherein like reference numerals designate identical or corresponding parts throughout the several views, FIG. 1a illustrates the anatomy of an abdominal aortic aneurysm (2) after a modular stent graft implantation and its anatomic juxtaposition of the inferior vena cava (3) to the aortic aneurysm sac (2). The stent graft pieces 5a and 5b are joined in the stent overlap zone 4. Element (d) corresponds to the distance between the inferior vena cava (3) and the aneurysm sac (2), which can vary in different individuals.

FIG. 1b illustrates the same anatomy and illustrates the potential sources of endoleaks; items 1a and 1b are the proximal (i.e. upstream) and distal (i.e. downstream) landing zones of the stent graft, respectively, where eponymously numbered endoleaks can occur (type 1a and 1b). The inferior mesenteric artery (6), the lumbar arteries (7,) or intercostal arteries (not shown) are other common source of endoleaks (type 2). Type 3 endoleaks arise from poor sealing at the overlap zone of the stents (4). Type 4 endoleaks are the result of transfabric flow.

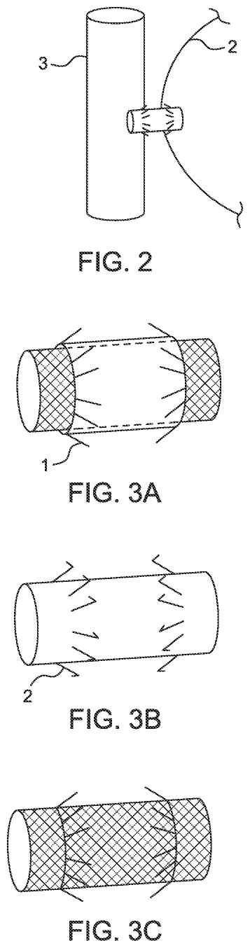

FIG. 2 illustrates one potential version of the device, which is deployed and connects the IVC and the aortic aneurysm sac. In this version the device is comprised of a cylindrical flow channel and incorporates integrated external reverse oriented barbs on either side to prevent slippage. This iteration of the device has a “one-piece” simple design and is symmetrically built, allowing implantation from the aortic side through a direct sac puncture or from the inferior vena cava side via a percutaneous approach.

FIG. 3a illustrates the device, which has an open cell configuration at its ends and has a covered middle section. The barbs preventing slippage are shown (1).

FIG. 3b illustrates the device with a fabric covered configuration. Note that the barbs (2) may have a small hook integrated at the distal end to prevent migration of the device once implanted.

FIG. 3c shows an uncovered device with bare metal stent scaffolding exposed.

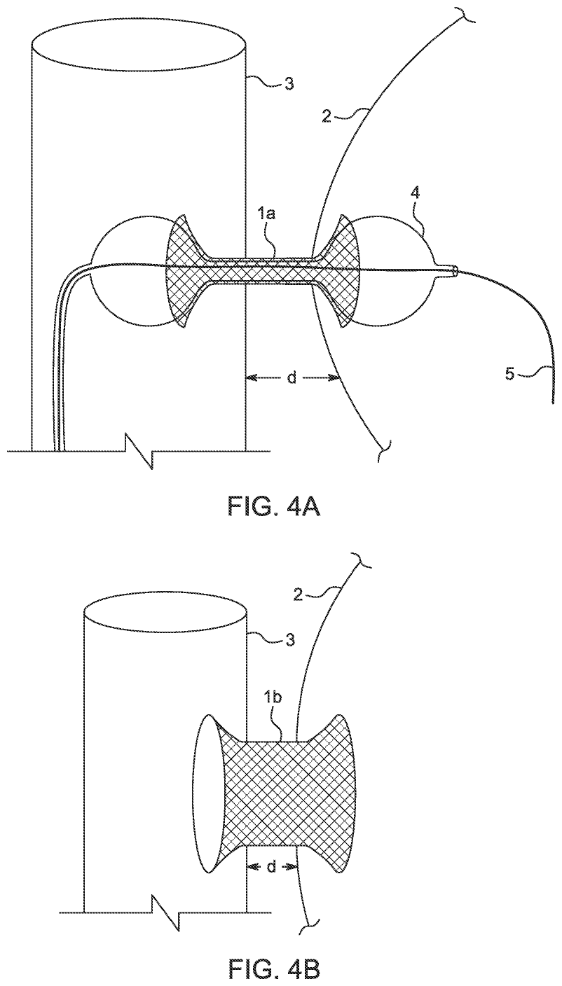

FIG. 4a is a view of a partially deployed balloon expandable device (la) connecting a large vein such as the inferior vena cava or another adjacent large vein to the aneurysm sac. The device is not fully expanded in this view and the balloon at the end of a catheter (4) is being inflated to expand the device.

FIG. 4b illustrates the fully expanded device (1b). In this iteration, the device is designed with a funnel shape at both ends. Note that the balloon expandable version of the device shortens as it is expanded, which aids in the apposition of the aneurysm sac and the vein wall by shortening distance (d) in FIG. 4b.

FIG. 5a, b, and c illustrate a device with a filter (6) and reverse barbs (7) that include an integrated screw off wire-release mechanism (8). Once the device is positioned, a release mechanism such as clockwise (or counterclockwise depending on orientation) wire rotation results in screw mediated disconnection of the deployment wire (8a) from the device.

In FIGS. 5b and 5c, an iteration of the device has a plurality of rows (2 rows are shown) of reverse barbs and a “donut” (toroid) shaped thickening, in this case, behind the funnel on the aortic end. The steps of deployment of a device with an integrated toroid sealing ring is shown in FIG. 5b, the distance (d) between the inferior vena cava and the aortic sac is reduced as traction (5b, arrow) is exerted on the integrated wire. In FIG. 5b as wire traction is increased, the second row of barbs is pulled into the Vena Cave. The additionally advanced row of barbs helps to maintain the apposition between the aorta (2) and vena cava (3), reducing the distance (d), resulting in improved seal. The traction wire (8a) can then be release via its integrated mechanism, which may be screw mediated (as described above) or which may comprise hook, magnet, or other mechanisms.

FIG. 6 a-c illustrates a device configuration with two components, which initially a device with a flat flange on the venous side and reverse is deployed from the IVC side. This first component (8) can have a rippled texture (FIG. 6a, 5) or multiple small barbs (FIGS. 6b, 7) to prevent back slippage. The large introducer sheath (9) can be used to hold the first component in place while deploying the second component. The introducer sheath can also be used to push the first component flush against the vein wall, if there is too much protrusion of the device into the venous system after initial deployment. In FIG. 6b a second component of the device is partially deployed with a smaller sheath going through the first component. FIG. 6b shows the second component partially released on the aneurysm sac side with a funnel or umbrella shaped end. After release of the umbrella shaped end in the aortic sac, the device is then pulled back towards the aortic sac wall (FIG. 6b, arrow) and subsequently fully released. This allows a complete seal between the 2 blood vessels except for the channel inside the device. FIG. 6c shows both components fully deployed. Note that the reverse funnel or umbrella shape on the aneurysm sac side prevents embolism.

FIG. 7 shows ‘on face’ (0°, a 45° and a side-view (90°) of a funnel shape end of the device which can be placed on the venous or aneurysm sac side.

FIG. 8 illustrates that in some circumstances it may be advantageous to have one or more filters integrated with the device. The filters can serve to strain debris and thrombus from the diseased arterial side, thereby preventing complications such as pulmonary emboli. In additional iterations filters can serve as flow regulators.

FIGS. 8a and 8b demonstrate other iterations of the device with conical filters at either end. FIG. 8a shows a device iteration with a filter on both ends of a device with an integrated sealing ring. FIG. 8b demonstrates a device with a filter and sealing ring on both ends. In FIG. 8c, rows of reversed barbs and filters are present. It should be noted that the filters may be designed to protrude from the ends of the device cylinder, or may be contained within the cylinder as illustrated in 8d.

FIG. 8d demonstrates the device with a conical filter inside the cylindrical portion of the device.

FIG. 8e demonstrates another itineration of the device with a conical filter inside the device, but in a different position. The position of the integrated filter may be important in the ability to re-access the lumen, or clear the device of debris at a later date.

The filters illustrated in FIGS. 8a-e can be open cell or partially or fully cloth covered, in which case the filter at the same time works as a flow-reducing valve, as it can also down regulate the blood flow between the aneurysm sac and the large vein. Any of the iterations 8a-e may incorporate release or traction wires as illustrated in FIG. 5.

FIG. 9 a-d demonstrates configurations of the one and two component device with a filter and flow reducing valves. FIG. 9a shows a filter with a conical shape towards the aneurysm sac side, to prevent embolism. Inside the device there is a flow reducing conical valve.

FIG. 9b shows a similar configuration with the valve component inside the cylindrical part of the stent.

FIGS. 9c and d show the filters and a valve in different configurations and positions of the umbrella or conical end of the device.

FIG. 10 illustrates a 2-component device, where the aortic sac component with an umbrella shaped end (4) gets deployed first and the second component (5) with a long flange is then placed inside component 4. If the distance d is short, as in FIG. 10b, the inner cylindrical component of the second device component is then protruding further into the aneurysm sac (2). If the distance d is larger as in FIG. 2a, it will protrude less. This configuration allows for using the same device with different anatomical configurations and distances d and at the same time assures that on the venous side there is minimal protrusion of the device. FIG. 10a illustrates a larger distance d; FIG. 10b illustrates a small distance d.

FIG. 11a shows a cylindrical plug (4) with a central lumen for closure of the cylindrical portion of the device. This plug can be used to close the arterio-venous connection if required.

FIG. 11b shows a cylindrical plug with a funnel shape towards a central lumen (5) for closure of the cylindrical channel of the device. This plug can be used to close the arterio-venous connection, if required. The funnel like configuration will allow aid in future transvenous wire access to the aneurysm sac should the necessity arise.

FIG. 11c shows a cylindrical plug (6) with a central lumen for closure of the cylindrical portion of the device. This plug is placed near the aneurysm sac end of the device, and should access to the aneurysm sac be needed in the future or should re-establishment of the communication be required, the plug can be dislodged and pushed into the aneurysm sac using conventional wires, catheters, or balloons.

FIG. 11d shows a device with reversal of the filter direction.

The potential device designs are not limited to those illustrated in the disclosure. Any practitioner of average skill in the art may produce other designs variations of substantially similar function. The device may be comprised of a variety and plurality of materials, including, but not limited to metals, fabrics, and plastics. Associated features may include plugs, toroids, hooks, magnets, screws, among others, for the purposes of attachment, seal, ease of access, deployment, or retrieval. In addition to filters and flow restrictors, the device may integrate other designs which may aid in augmenting, restricting, or eliminating blood flow.

Obviously, numerous modifications and variations of the present invention are possible in light of the above teachings. It is therefore to be understood that within the scope of the appended claims, the invention may be practiced otherwise than as specifically described herein.

1. A device configured to be implanted to create a temporary or permanent connection between two hollow structures, comprising:
a sent configured to be disposed between an aortic or large vessel aneurysm sac and a large adjacent vein.

2. The implantable device according to claim 1, wherein the stent is either balloon expandable or self-expandable.

3. The implantable device according to claim 1, wherein the stent is either an open cell stent without covering, or the stent is partially or fully covered with fabric or film.

4. The implantable device according to claim 1, wherein the stent includes one or a plurality of filters at one or both ends thereof or within the cylindrical portion thereof.

5. The implantable device according to claim 1, comprising two or more stents that can be joined to bridge different distances between the aneurysm sac and the vein.

6. The implantable device according to claim 1, wherein one or both ends of the stent are conical, funnel shaped, tapered, or flanged.

7. The implantable device according to claim 1, wherein a conical or umbrella shaped end of the stent is attachable to either end or both ends of the aortic sac or the venous side.

8. The implantable device according to claim 1, wherein a flow-reducing valve or a plurality of valves is disposed at one or both ends of the stent, or within the stent.

9. The implantable device according to claim 1, wherein at least a portion of the stent has at least one of a textured surface, a plurality of barb rows, a plurality of donuts, toroids, or undulations.

10. The implantable device according to claim 4, wherein the one or a plurality of filters may be flat, dome shaped or conical, oriented antegrade or retrograde to flow, and may be integrated or detachable.

11. The implantable device according to claim 10, wherein the one or a plurality of filters is open cell, wire mesh or partially or completely fabric covered.

12. The implantable device according to claim 1, wherein the stent includes at least one conical, tapered, or flared end to facilitate future access to the aneurysm sac.

13. The implantable device according to claim 1, wherein the stent is configured to be closed or partially closed with a plug to reduce flow in the arterio-venous connection.

14. The implantable device according to claim 1, wherein the plug is configured to stop the flow in the arterio-venous connection.

15. The implantable device according to claim 14, wherein the plug includes a funnel or conical shape on one or both sides, tapering to a wire-accessible central lumen.

16. The device of claim 15, wherein at least one of the stent and the plug are composed of a material including at least one of metal, fabric, plastic, or films.

17. The device of claim 16, wherein the material is dissolvable.

18. The device according to claim 17, wherein the material is fully dissolvable.
